(19) United States
(12) Patent Application Publication
(10) Pub. No.: US 2024/0182797 A1
(43) Pub. Date: Jun. 6, 2024

(54) INTEGRATED PROCESS FOR PRODUCTION OF CHEMICAL FROM CRUDE OIL USING SLURRY HYDROCRACKING

(71) Applicant: Indian Oil Corporation Limited, Mumbai (IN)

(72) Inventors: Nayan DAS, Faridabad (IN); Mainak SARKAR, Faridabad (IN); Sarvesh KUMAR, Faridabad (IN); Ramesh KARUMANCHI, Faridabad (IN); Madhusudan SAU, Faridabad (IN); Sankara Sri Venkata RAMAKUMAR, Faridabad (IN)

(73) Assignee: Indian Oil Corporation Limited, Mumbai (IN)

(21) Appl. No.: 18/526,544

(22) Filed: Dec. 1, 2023

(30) Foreign Application Priority Data
Dec. 2, 2022 (IN) 202221069702

(51) Int. Cl. C10G 47/22; C07C 4/04

(52) U.S. Cl. CPC C10G 47/22; C07C 4/04; C10G 2300/1059; C10G 2400/20; C10G 2400/30

(57) ABSTRACT

The present invention relates to an integrated process for conversion of crude oil to petrochemicals viz. olefins and aromatics. More specifically, the present invention provides a configuration of processes both primary and secondary processing units through which the crude oil can be converted to petrochemicals. The configuration of processes provided by the present invention utilizes minimum numbers of secondary processing units and provides great degree of flexibility towards maximizing yield of either petrochemical or fuels depending upon market scenario.

### CROSS-REFERENCE TO RELATED APPLICATIONS

This application claims the benefit of priority of Indian Patent Application No. 202221069702 filed on Dec. 2, 2022, the disclosure of which is incorporated herein by reference in its entirety.

### FIELD OF INVENTION

The present invention is related to a noble process configuration for conversion of crude oil to petrochemicals viz. olefins and aromatics. More specifically, the present invention discloses a noble configuration of processes both primary and secondary processing units through which the crude oil can be converted to petrochemicals. Further, this innovative configuration of processes utilizes minimum numbers of secondary processing units and provides great degree of flexibility towards maximizing yield of either petrochemical or fuels depending upon the market scenario.

### BACKGROUND OF THE INVENTION

Demand of petrochemicals is increasing with increase in global population and GDP. Presently major portion of crude oil is being used for producing fuels. However, due to the factors like strict environmental regulations, low fuel demand growth due to global drive towards energy system decarbonization; the refiners have started facing challenges and seeking avenues for altering their product pattern from fuels to petrochemicals and other non-fuel products. With the present available technologies and refinery petrochemical integration, only 10-15 wt % of the crude oil can be converted to petrochemical. Refiners are now looking forward towards dedicated crude oil to chemical complexes; where, at least 40 wt % of the crude oil can be converted into petrochemicals. Different concepts of processing crude oil to chemicals have been explored by various researchers and organizations in last one decade. However, challenges are still there with respect to number of processing steps, CAPEX, OPEX, flexibility w.r.t crude selection and product pattern etc.

US20180142166A1 reveals an integrated process and system for converting crude oil to petrochemicals and fuel products. The designs utilize minimum capital expenditures to prepare suitable feedstocks for the steam cracker complex. This prior art discloses an integrated configuration of processing units for converting crude oil to petrochemical, and fuel products, using mixed feed and gasoil steam cracking units. The other process units in this configuration are atmospheric and vacuum distillation unit (AVU), diesel hydrotreating unit, kerosene sweetening unit, VGO hydrocracking unit and residue treating unit. The mixed feed steam cracking unit can process naphtha streams with boiling range between 20 and 205° C. In certain embodiments of this prior art, processing of mixed feed steam cracker products in series of units like butadiene extraction unit, MTBE unit and metathesis unit has been also disclosed. This prior art further discloses that product from gasoil steam cracker is processed in aromatic extraction unit and transalkylation unit to produce BTX rich streams.

US20180142167A1 describes the process and system for the conversion of crude oil to chemicals and fuel products integrating steam cracking and fluid catalytic cracking in which addition to the process described in US20180142166A1. Fluidized catalytic cracking unit is adopted in the scheme for production of olefinic feedstock particularly propylene. The steam cracker in this configuration is a mixed feed steam cracker which can process naphtha boiling range streams. Other processing units are similar as that of US20180142166A1.

US 2018/0155633 A1 describes about the process scheme that enable conversion of crude oil feeds with several processing units in an integrated manner into petrochemicals. The integration of processes disclosed in this prior art includes mixed feed steam cracking and gas oil steam cracking. Other units include Atmospheric distillation unit, vacuum distillation unit, gas oil Hydrocracking unit, delayed coker unit, diesel hydrotreater unit, butadiene extraction unit, 1-butene recovery unit, MTBE unit, pyrolysis gas hydrotreatment and recovery unit. The crude oil conversion achieved in this conversion claimed to be in the range of 39-80 wt %.

US 2018/0155639 A1 discloses another integration scheme of processing units similar to US 2018/0155633 A1 with addition of another process unit for conversion of Vacuum residue into Naphtha which along with unconverted oil is sent into steam cracking zone and middle distillate stream to gasoil Hydrocracking zone. Also, Base oil production unit is added to produce the base oil products and distillates from the unconverted oil fraction from the gas oil Hydrocracking unit.

U.S. Pat. No. 10,167,434 discloses an integrated hydrocracking process for production of olefinic and aromatic petrochemicals from a hydrocarbon feedstock from crude oil. The processing steps comprises of multiple hydrocracking units and gas cracking section. Crude oil is directly processed in a crude oil hydrocracker along with other streams like residue from other hydrocracking sections. The heavier part from crude hydrocracking is further processed in a second hydrocracking zone and the slurry part from second hydrocracking zone is processed in slurry hydrocracking unit. The second hydrocracking zone comprises of one or more hydrocracking units for processing streams like gasoline, gas oil, aromatic ring opening etc. The lighter gas generated in this process is separated in separation section and further thermally cracked to produce olefin and aromatic rich product streams. The scheme also consists of C4 dehydrogenation and olefin purification section. This process claims to have significantly high LPG yield.

All the literature described above have similar objective of enhancing chemical yield from crude oil. Most of the processes mainly focus on minimizing bottom of the barrel of crude oil through hydroprocessing. Olefin production in these processes is through thermal cracking of light/middle distillates. Steam crackers based different feed stock is being explored by different researchers. The gas-based steam cracker is always preferable for high ethylene yield whereas naphtha and gas oil-based steam cracker gives high C3 and C4 olefins along with aromatic rich pyrolysis gasoline. Therefore, choosing suitable configuration based on suitable unit and feedstock is vital in any crude to chemical configuration. On the other hand residue upgradation through hydrocracking is critical to improve lighter production and loss of hydrocarbon in form of coke. Multiple numbers of hydrocracking units in the configurations have been explored in different studies and most of them are found to be useful in improving chemical yield. However, for selection of proper configuration for Crude to Chemical one must look into following points:
i. Configuration should be of low CAPEX intensive
ii. Configuration should be flexible in producing Chemical and fuel based on market demand
iii. Even within petrochemical product, demand pattern of olefins and aromatics changes. Therefore, complex must be flexible in re-routing naphtha to naphtha cracker/aromatic complex for production of olefins or aromatics.
iv. Crude to chemical configuration through revamping of existing crude processing units increases adoptability of the configuration.

### OBJECTIVES OF THE INVENTION

It can be inferred from the prior art that need still exists for an efficient integration of processes and processing unit for optimal conversion of crude oils into chemicals.

Accordingly, primary objective of the present invention is to provide a noble configuration of processes both primary and secondary processing units through which the crude oil can be converted to petrochemicals.

Another objective of the present invention is to provide an innovative configuration of processes utilizes minimum numbers of secondary processing units and provides great degree of flexibility towards maximizing yield of either petrochemical or fuels depending upon market scenario.

### SUMMARY OF THE INVENTION

Accordingly, the present provides a process for complete conversion of crude oils by integrating atmospheric and vacuum fractionation unit, selective mild hydrocracking unit, Residue Hydrocracking unit, naphtha cracker unit and aromatic complex. The process provided by the present invention is very flexible in terms of chemical and fuel production from crude oil.

The present invention provides an integrated process for converting crude oil to high value petrochemicals products, comprising:
i. fractionating crude oil in an atmospheric vacuum unit (AVU) comprising an atmospheric distillation column and a vacuum distillation column to obtain straight run fractions comprising, a straight run naphtha (SRN) having boiling range of C5-200° C., a middle distillate stream having boiling range of 200 to 370° C., a vacuum gas oil (VGO) with boiling range of 370 to 560° C. and a vacuum residue (VR) having boiling range above 560° C.;
ii. routing vacuum residue to a Residue Hydrocracking section comprising a slurry hydrocracking reactor and a gas-oil reactor to obtain a stream comprising fuel gas in the range of C1 and C2), liquefied petroleum gas (LPG) in the range of C3 and C4, total naphtha having boiling point in the range of C5 to 200° C., middle distillate having boiling point in the range of 200 to 370° C. and unconverted Pitch having boiling point above 560° C. by slurry hydrocracking;
iii. routing the total naphtha towards a naphtha cracker unit for production of olefins;
iv. routing the middle distillate stream towards a selective mild hydrocracking unit;
V. routing the VGO partly towards the gas-oil reactor of the Residue Hydrocracking section;
vi. routing the middle distillate stream from atmospheric distillation unit towards a selective mild hydrocracking unit to obtain a stream comprising fuel gas, LPG, light naphtha having boiling range between C5-100° ° C., heavy naphtha having boiling range between 100 to 200° ° C. and diesel stream having boiling point above 200° ° C.;
vii. routing the light naphtha towards a naphtha cracker unit;
viii. routing the heavy naphtha towards an aromatic complex comprising reformer, transalkylation unit, isomer unit, p-xylene extraction unit and aromatic extraction unit;
ix. processing the heavy naphtha in the aromatic complex to obtain hydrogen gas, off gas, LPG, non-aromatic raffinate, benzene, p-xylene and heavy aromatics;
x. routing the SRN towards a naphtha cracker unit to obtain a stream comprising ethylene rich fuel gas stream, C3 and C4 olefin rich mixed stream, monoaromatic rich pyrolysis gasoline stream and pyrolysis fuel oil stream;
xi. routing the ethylene rich fuel gas stream towards a C2-splitter unit to obtain ethylene and fuel gas;
xii. routing the C3 and C4 olefin rich mixed stream towards a propylene recovery unit to obtain propylene, 1-butene, butadiene and LPG;
xiii. routing the monoaromatic rich pyrolysis gasoline stream towards the aromatic complex to obtain benzene and p-xylene;
xiv. routing the pyrolysis fuel oil stream towards Residue Hydrocracking section to crack further.

Present invention further provides an integrated system for converting crude oil to high value petrochemicals products, comprising:
(i) an atmospheric vacuum unit (AVU) comprising an atmospheric distillation column and a vacuum distillation column to fractionate crude oil into straight run fractions comprising, a straight run naphtha (SRN) having boiling range of C5-200° C., a middle distillate stream having boiling range of 200 to 370° C., a vacuum gas oil (VGO) with boiling range of 370 to 560° C. and a vacuum residue (VR) having boiling range above 560° C.;
(ii) a Residue Hydrocracking section comprising a slurry hydrocracking reactor and a gas-oil reactor to receive and process the vacuum residue to produce a stream comprising fuel gas in the range of C1 and C2), liquefied petroleum gas (LPG) in the range of C3 and C4, total naphtha having boiling point in the range of C5 to 200° ° C., middle distillate having boiling point in the range of 200 to 370° C. and unconverted Pitch having boiling point above 560° C. by slurry hydrocracking, wherein,
   a naphtha cracker unit operable to routing the total naphtha for production of olefins;
   a selective mild hydrocracking unit operable to routing the middle distillate stream; and
   the gas-oil reactor of the Residue Hydrocracking section operable to partly routing the VGO,
(iii) a selective mild hydrocracking unit to receive the middle distillate stream from the atmospheric distillation unit and process the said stream to produce a stream comprising fuel gas, LPG, light naphtha having boiling range between C5-100° C., heavy naphtha having boiling range between 100 to 200° ° C. and diesel stream having boiling point above 200° ° C., wherein, the light naphtha is routed towards a naphtha cracker unit;
(iv) an aromatic complex comprising reformer, transalkylation unit, isomer unit, p-xylene extraction unit and aromatic extraction unit, wherein the aromatic complex receives heavy naphtha and process said heavy naphtha to produce hydrogen gas, off gas, LPG, non-aromatic raffinate, benzene, p-xylene and heavy aromatics;
(v) a naphtha cracker unit to receive SRN and process said SRN to produce a stream comprising ethylene rich fuel gas stream, C3 and C4 olefin rich mixed stream, monoaromatic rich pyrolysis gasoline stream and pyrolysis fuel oil stream, wherein,
   the aromatic complex operable to routing the monoaromatic rich pyrolysis gasoline stream to produce benzene and p-xylene;
   Residue Hydrocracking section operable to routing the pyrolysis fuel oil stream to crack further.

(vi) a C2-splitter unit to receive the ethylene rich fuel gas stream and process the said stream to produce ethylene and fuel gas; and

(vii) a propylene recovery unit to receive the C3 and C4 olefin rich mixed stream and process.

The integrated process and system disclosed in the present invention can convert at least 45% of crude oils into chemicals.

### BRIEF DESCRIPTION OF THE DRAWINGS

FIG. 1 provides the process flow diagram of the present invention.

FIG. 2 provides the process flow diagram of the processes taking place inside the residue hydrocracking unit (36) of the present invention.

### DETAILED DESCRIPTION OF THE INVENTION

While the invention is susceptible to various modifications and alternative forms, specific embodiment thereof will be described in detail below. It should be understood, however that it is not intended to limit the invention to the particular forms disclosed, but on the contrary, the invention is to cover all modifications, equivalents, and alternative falling within the scope of the invention as defined by the appended claims.

The following description is of exemplary embodiments only and is not intended to limit the scope, applicability, or configuration of the invention in any way. Rather, the following description provides a convenient illustration for implementing exemplary embodiments of the invention. Various changes to the described embodiments may be made in the function and arrangement of the elements described without departing from the scope of the invention.

This present invention describes about a flexible configuration scheme for converting crude oil into petrochemical. Using minimum numbers of processing units, this configuration can produce maximum amount of petrochemical. This present process also flexible in terms of fuel and petrochemical production and can be configured according to the requirement.

In this present invention, ‘crude oil’ defined as mixtures of hydrocarbon obtained from earth crust and that have undergone some pre-treatment such as water-oil separation; and/or gas-oil separation; and/or desalting; and/or stabilization.

The term ‘LPG’ defined in this invention are those saturated hydrocarbons having carbon no. C3 and C4.

‘SRN’ is the acronym of Straight Run Naphtha generating from atmospheric distillation column and have a boiling range between C5-180° C.

‘Light Naphtha’ is a hydrocarbon stream generates from distillation of petroleum fraction and have boiling range between C5-100° C.

‘Heavy Naphtha’ is another hydrocarbon stream having boiling range between 100-200° C. generates from distillation of petroleum fraction.

The term ‘TN’ is the acronym of Total Naphtha stream and defined as mixture of ‘light naphtha’ & ‘heavy naphtha’ obtained from Residue Hydrocracking Section.

‘MD’ is the acronym of Middle Distillate stream in this invention refers as a distillate stream having boiling range between 180 to 370° C.

‘SR-MD’ is the acronym of Straight Run Middle Distillate stream in this invention refers as a distillate stream having boiling range between 180 to 370° C. originating from atmospheric distillation column.

‘SR-VGO’ is the acronym of Straight Run Vacuum Gas Oil having boiling range between 370-560° C., generates from Vacuum distillation column of atmospheric and vacuum unit.

‘VGO’ is the acronym of Vacuum Gas Oil having boiling range between 370-560° C., generates from Vacuum distillation column other than AVU unit.

‘VR’ is another acronym of Vacuum Residue used in this literature and have a boiling range between (560° C.+) generating from vacuum distillation column.

The term ‘Heart Cut’ is defined as a naphtha stream generating from Selective Mild Hydrocracking Unit and have a boiling range between 100-200° ° C.

‘Pyrolysis gasoline’ is defined as stream generating from naphtha cracker unit having boiling range between C6-200° ° C.

‘PFO’ is the acronym of Pyrolysis Fuel Oil having initial boiling point of 200° C., generates from Naphtha Cracker Unit.

‘Pitch’ defined in this invention is an unconverted semi solid stream rich in resins and asphaltenes.

Other streams and units used in this entire literature are well known in the art of refinery and petrochemicals and used for usual purposes.

The present crude oil processing configuration consists following units/section.
i. Atmospheric & Vacuum Unit (AVU)
ii. Residue Hydrocracking Section
iii. Selective Mild Hydrocracking Unit
iv. Naphtha Cracker Unit (NCU)
v. Aromatic Complex
vi. C2 splitter
vii. Propylene Recovery Unit (PRU)
   i. Atmospheric & Vacuum Unit (AVU):

This is the combination of distillation column designed to fractionate hydrocarbons present in the crude oil. The main purpose of this unit is to fractionate crude oil based on the boiling range. This unit consists of atmospheric column and vacuum column. Crude oil is generally charged in atmospheric distillation column. Five streams namely LPG, SRN, SR-MD, SR VGO and VR are generated from this section. The atmospheric column fractionates crude oil and produces LPG, SRN, SR-MD. Vacuum distillation column takes bottom heavier part from atmospheric column and further fractionate into VGO and VR. VGO is generally routed towards FCC or HCU/OHCU unit for further cracking to produce fuels. However, in the present embodiment, VGO & VR both are processed in the Residue Hydrocracking Section. The atmospheric distillation unit operating at a pressure in the range of 1 to 2 bar (g) and top temperature in the range of 150 to 250° C., preferably in the range of 190 to 210° C. The vacuum distillation unit operates at pressure in the range of 0.01 to 0.05 bar(g).

ii. Residue Hydrocracking Section

This section is most critical section of the process. This section is the combination of two hydrocracking unit namely slurry hydrocracking and gas-oil hydrocracking unit. Slurry phase hydrocracking is an emerging and capable technology for achieving a high conversion of heavy feedstocks like VR and minimizing byproducts such as gas, coke, and fuel oil. The reason behind choosing slurry hydrocracking over other conventional VR processing units e.g., ebulated bed, delayed coker etc are as follows.
a) slurry unit is capable of handling of VR of any CCR and also can process any type of crude oil.
b) High valued distillate stream can be produced.
c) Pitch generated in this process can further processed in the bitumen unit to produce bitumen of VG 30 &40 grade.
d) Unlike coker unit hydrocarbon molecules is being preserved in the process for further processing in other secondary conversion units.

In the present invention, VR and PFO are used as feedstock for slurry hydrocracking unit. The unit consists of slurry reactor operated on upflow mode. Liquid catalyst with Group-IV and Group-V metal function is used in the slurry reactor. Ex.-slurry product is being fractionated to generate streams like LPG, TN, MD and VGO. TN is one of the feedstocks for NCU and MD is being processed in the selective mild hydrocracking unit. VGO produced in this section is further processed in gas-oil hydrocracking unit located downstream of the slurry hydrocracking unit. VGO is mixed with SR-VGO and hydrocracked in this unit to produce more amounts of TN and MD. The slurry hydrocracker unit operates at a pressure in the range of 45 to 80 bar(g), preferably in range of 50-70 bar (g) and at temperature in the range of 360 to 450° C., preferably in range of 390 to 420° C.

The ex. Slurry product from the reactor further hydrocracked in the gas-oil hydrocracking unit located in the downstream of the slurry reactor. Feedstock of this unit includes VGO generating from slurry hydrocracking and SR-VGO generating from AVU. This unit consists of two fixed bed reactors operating in downflow mode and loaded with conventional hydroprocessing catalysts. The first reactor of the unit is loaded with hydrotreating catalyst, and second reactor is with hydrocracking catalyst. VGO is treated in the first reactor to remove nitrogen and other impurities. Hydrotreated product is therefore hydrocracked to produce lighter hydrocarbons. Hydrocracked product is therefore fractionated in the distillation column located within this section. Pitch is taken out of the system as bleed and can be further utilized to product bitumen of VG 30 & 40 grade. The naphtha stream produced from ex-slurry hydroprocessing unit has sulfur content <0.5 ppmw. The reactors of this unit operate at a pressure in the range of 45 to 150 bar(g), preferably in range of 50-120 bar (g) and temperature in the range of 360 to 450° C., preferably in range of 370 to 420° C.

iii. Selective Mild Hydrocracking Unit (SMHC):

Feedstock of this unit is any kind of middle distillate having boiling range of 180-420° C., more specifically 200-380° C. This unit is specially designed to produce heavy naphtha (HN) with very high N+2A value (N: Naphthene and A: Aromatic) for Continuous Catalytic Reforming Unit (CCRU) and light naphtha (LN) for NCU. This unit can process any kind of cracked middle distillate e.g., Light Cycle Oil (LCO) & Coker Gas Oil (CGO). In fact, cracked stream is best for the unit to increase mono-aromatic concentration of heavy naphtha. The process utilizes mild hydrocracking catalyst to selectively convert di-aromatic and poly aromatic hydrocarbon to produce monoaromatics. The unit consists of two reactors and product fractionation section. The first reactor of the system is for hydrotreating purpose. Aromatic rich middle distillate stream is treated in the presence of hydrogen to remove heteroatoms. Also, in this reactor aromatic side rings of Di- and PAH aromatics get saturated by hydrogen leaving one aromatic ring intact. Hydrotreated effluent is further sent to the second reactor loaded with mild hydrocracking catalyst. Strong acid functionality of mild hydrocracking catalyst opens up naphthenic side ring of the aromatic compounds and also breaks down paraffinic compounds into smaller paraffin. Isomerization of paraffins also takes place in this reactor as side reaction increasing i-paraffin content in the product. The hydrocracked product is therefore splitted into product fraction through fractionator located at the downstream of the reactor. Three main product cuts are being generated in this unit and they are Light naphtha (LN) having boiling range of C5-100° C., heavy naphtha (HN) having boiling range of 100-200° C. and diesel having minimum boiling point of 200° C. LN produced in this process contains high i-paraffins and exhibits high RON of at least 82. Also, LN produced in the process has very low sulfur content (S<0.5 ppmw). This product can either be routed to NCU for production of olefins or to the gasoline blending pool. HN produced in this process routed towards CCRU. In common practice CCRU intakes straight run naphtha having boiling range between 100-200° C. This straight run naphtha has very low N+2A content (range: 60-65) [N: naphthene content, A: aromatic content]. N+2A is the quality parameter used to evaluate feed quality of CCRU. Naphtha with high N+2A value reduces furnace duty of the reformer unit and enhances catalyst life. HN produced in this process have N+2A value of at least 70 units and can therefore substitute straight run naphtha and replaced naphtha can be routed towards NCU for production of olefins. Also, HN produced through the process has low sulfur content (S<0.5 ppmw) and can be directly fed into CCRU. No hydrotreating reactor is required to remove sulfur, which is detrimental to the platinum catalyst used in CCRU. Diesel is the third stream produced in this process. Sulfur content of the diesel can be as low as 10 ppmw depending upon the feedstock handled in this unit. Therefore, diesel can be blended to the pool. Otherwise, need further hydrotreatment to produce diesel of EURO-VI/BS-VI quality.

In the present invention, SR-MD produced from AVU and MD stream produced from residue hydrocracking section are mixed together and processed in SMHC unit to produce LN, HN and Diesel. LN produced in this process is routed towards NCU unit. HN is further processed in aromatic complex to produce p-xylene or BTX (benzene, toluene & xylene). Diesel stream is taken out as product.

In one of the embodiments, it is disclosed that LN can be blended with gasoline boiling streams generating from other units to produce Gasoline.

In another embodiment, it is further disclosed that some part of diesel can be recycled back to the reactor to increase naphtha yield. The part of diesel stream produced from selective mild hydrocracking unit is recycled back into the first reactor of the unit with a fresh feed to recycle ration of 90:10, more favorably 70:30 ration. Operating this unit in such recycle mode decreases hydrogen consumption and LPG generation of the process.

In yet another embodiment, it is disclosed that reactors of this unit are operated at a pressure in the range of 45 to 100 bar(g), preferably in the range of 50 to 100 bar(g) and at temperature in the range of 350 to 420° C., preferably in the range of 360 to 410° C.

iv. Naphtha Cracker Unit (NCU):

This unit is a thermal cracking unit. Feedstock of this unit is naphtha diluted with steam and therefore often called naphtha cracking or steam cracking. Thermal cracking is largely used for the production of olefins. Wide range of hydrocarbon feedstock can be processed in steam cracker. Steam cracker based on lighter feedstock e.g., ethane propane is common in USA due to abundant presence of natural gas. Rest of the world is mostly depending upon petroleum lighter cuts like naphtha. Heavier streams like gas oil and even crude oil can be processed in the steam cracker. However, yield pattern largely depends upon the type of feedstock handled in the steam cracker. For example, with ethane is best feedstock for producing lower olefins. Ethylene to propylene ratio (C2=/C3=) decreases steadily as feedstock shift from ethane to gas oil. Similarly heavy feedstock increases pyrolysis gasoline production of the unit. Therefore, naphtha as feedstock gives balance yield of C2 & C3 olefins through pyrolysis.

In the present invention, Straight run naphtha from AVU, TN from Residue Hydrocracking unit and LN from SMHC unit has been processed in NCU to produce ethylene, propylene, pyrolysis gasoline, fuel oil (PFO) and other heavy olefins and hydrocarbons. Ethylene rich C2 hydrocarbon stream produced in this unit is sent to C2 splitter where ethane and ethylene gets separated. Propylene rich LPG stream is processed in the propylene recovery unit. Pyrolysis gasoline consists of 60-80% aromatics, specially C6-C9 aromatics. Therefore, this stream is valuable feedstock for aromatic complex and processed in aromatic complex for production of p-xylene. Fuel oil produced in the naphtha cracker is mixture of heavy molecules like naphthalene, polycyclic aromatic hydrocarbons. Fuel oil is routed towards Residue Hydrocracking section to process further.

In one of the embodiments, it is disclosed that the naphtha cracker unit operates at a reactor outlet temperature of 580 to 670° C., preferably between 590 to 630° C., reactor pressure in the range of 0.7 to 2.5 bar(g), preferably in the range of 0.8 to 1.5 bar(g) and the catalyst to oil ratio in the range of 15 to 30, preferably in the range of 15 to 25.

In yet another feature it is disclosed that olefin production can be increased with moderate decrease in aromatic production by rerouting HN produced from selective mild hydrocracking into NCU. By processing heavier HN feedstock consisting of aromatics, the pyrolysis gasoline quantity increases and therefore aromatic production also increases. In this way olefin production can be optimized based on the product demand.

v. Aromatic Complex:

Aromatic complex is combination of units to produce aromatic building block molecule like BTX. Configuration of Aromatic complex depends upon the product demand, feedstock and most importantly capital investment. The simplest complex is only aimed at the production of benzene, toluene, and mixed xylenes (BTX). Some of the complex is only aimed at the production of p-xylene. In that case, the complexity increases as number of units to be added for the conversion of the isomers and to increase product purity Feedstock consideration is also an important parameter towards the selection of configuration. Aromatic Complex with reformer/CCRU takes naphtha with specific boiling range (90-200° C.) as feedstock. Some aromatic complex is specially designed to produce p-xylene. However, some amount of benzene is also produced inevitably through the process and separated out of the product and sold separately. Other product streams include hydrogen, LPG, raffinate and heavy aromatics. In a standard p-xylene complex (PX-complex) following unit must be there in order to achieve maximum p-xylene yield.
i. Naphtha Hydrotreating Unit
ii. Continuous catalytic reformer unit (CCRU)
iii. Aromatic Extraction unit
iv. Trans Alkylation Unit
v. p-xylene extraction unit
vi. Isomerization Unit
vii. Product purification section

Naphtha hydrotreating unit is for hydrotreatment of naphtha feed to remove sulfur, nitrogen and oxygen. These heteroatoms are detrimental for the CCRU catalyst therefore removed through hydrotreatment. Treated naphtha therefore processed in CCRU or simply reformer unit.

Naphtha reformer unit present in the aromatic complex aromatizes molecules to produce BTX rich reformate. Platinum catalyst based regenerative or semi-regenerative process unit can be used for this purpose. One of the key criteria of the platformer feed is N+2A content of the feed naphtha (N: amount of naphthene & A: amount of aromatics). Higher the values better the feedstock and also it contributes toward lower operating temperature for a particular conversion.

Aromatic extraction unit serves propose of separating aromatics from non-aromatics using solvent. It is most economic way for separating aromatics from non aromatics. A number of solvents like NMP, dimetylsulfooxide, diethylglycol, aniline, furfural etc. is commonly used in this process. Sulfolane, a popular solvent among the licensor used as solvent in extractive distillation process (Sulfolane process). This process can extract non aromatics from aromatics. Because of the higher selectivity towards aromatic separation, sulpholane units operate at the lowest available solvent-to-feed ratio for any given reformate feedstock. Therefore, a sulpholane unit is much cheaper in operation than any other type of extraction unit. In a modern, fully integrated aromatics complex, the sulpholane unit is located downstream of the reformate splitter column. The C6-C7 fraction from the overhead of the reformate splitter is fed to the sulpholane unit. The raffinate from the sulpholane unit is usually blended into the gasoline pool.

Transalkylation Unit is used in the complex to maximize the xylene quantity. Transalkylation process is very common route to convert toluene into mixed xylene. Tatoray Process is a catalytic conversion process where toluene is converted into benzene and xylene. UOP is the licensor of this process. In more practical scenario, the C8+ aromatic stream (A8+) stream along with toluene converted into A9+ stream. Two major reactions involved in the Tatoray process are disproportionation and transalkylation. In disproportionation reaction, two moles of toluene reacts and benzene along with xylene forms. Transalkylation is the reaction where toluene reacts with A9 aromatic molecule and forms xylene.

Para-xylene extraction unit is used to increase its purity. Higher degree purity is required in case of para xylene. Also, market value of para xylene is higher than the other isomers. Due to very close boiling point of the xylene isomers, separation through distillation is practically not possible. Therefore, adsorption is good option to increase purity of desired product. PAREX process is based on the adsorption process and it separates p-xylene from mixed C8 stream. The C8 stream is consist of o-xylene, meta-xylene and p-xylene and ethyl benzene stream. The adsorbent used in this process is solid zeolite. A typical PAREX unit consists of two adsorbent chambers/reactor in series. The rotary valve is used to switch the position of liquid feed in and withdraw of product. The dilute extract is sent to extract column and the dilute raffinate is sent to raffinate column. From the raffinate and extract column the diluents is recovered and fed into the bed through rotary valve. The overhead from the extract column is fed into a finishing column and highly pure p-xylene is drawn out. The raffinate overhead is mixture of C8+ hydrocarbons (o-xylene, m-xylene and ethyl benzene). The mixture is then sent to the isomerization unit where m-xylene and o-xylene converts into p-xylene and recycled back into PAREX unit.

In an Isomerization unit, xylenes and ethylbenzene converts into p-xylene through isomerization reaction. Isomerization of xylene normally occurs through the transfer of methyl group (i.e., conventional carbonium ion mechanism). Isomerization of ethyl benzene is a bit complex and presence of hydrogen is required.

Product purification section consists of multiple numbers of distillation columns to purify the product.

In the present invention, key target of the process is to produce petrochemical as well as fuel in flexible manner. Also optimized number of units has been used with its fullest potential in order to minimize CAPEX. For example, no naphtha hydrotreating unit is required in pre-reforming section of aromatic complex as all naphtha stream consists of sulfur <0.5 ppmw. Also, heavy naphtha from selective mild hydrocracking unit contains significant amount of aromatics which may get saturated in the hydrogen environment of hydrotreating.

One of the features of the process is one can increase fuel production at any point time by re-routing the LN stream generated from selective mild hydrocracking unit. Light naphtha stream can be used as blendstock for gasoline and will increase gasoline production.

According to another feature, SR VGO from AVU is co-processed with the VGO generated in slurry hydrocracking unit. Through this CAPEX of extra cracking unit can be minimized.

Another feature reveals that reformer unit located in Aromatic complex produces hydrogen during reforming of naphtha. Hydrogen is most costly stream in refinery. Therefore, this hydrogen can meet the hydrogen requirement at certain extent of hydroprocessing units of the configuration.

The present invention discloses an integrated process for processing crude oil to produce olefinic and aromatic feedstock for petrochemicals. The processing steps comprises of:
(i) Fractionation of crude oil in atmospheric and vacuum distillation unit named as ‘AVU’ to produce LPG, Total Naphtha, Middle distillate, SR-VGO and VR. Further processing of fractionated product is described as under.
(ii) Processing of Total Naphtha (TN) and LN stream generating from residue hydrocracking and Selective Mild Hydrocracking (SMHC) unit respectively in NCU to produce ethylene rich off gas stream, propylene rich LPG stream, high aromatic rich pyrolysis gasoline stream and heavy aromatic rich PFO stream.
(iii) Processing of SR-MD stream and MD stream from AVU and Residue Hydrocracking section respectively in a Selective Mild Hydrocracker Unit to produce LN suitable to use as feedstock in naphtha cracker complex, aromatic rich HN stream and diesel.
(iv) Processing of VR from AVU and PFO from NCU in a residue hydrocracking section comprises of slurry hydrocracking unit and gas-oil hydrocracking unit.
(v) Processing SR-VGO stream in gas-oil hydrocracking unit along with ex-slurry VGO.
(vi) Processing of Olefin rich off gas stream in C2-splitter to produce ethylene and off gas
(vii) Processing of propylene rich LPG stream in Propane recovery unit (PRU) to produce propylene, 1-butene and LPG.
(viii) Processing of HN stream from SMHC unit and pyrolysis gasoline stream in aromatic complex to produce chemicals (e.g., Hydrogen, benzene, p-xylene) and fuels (e.g., gasoline, diesel, LPG)

### DETAILED DESCRIPTION OF THE DRAWINGS

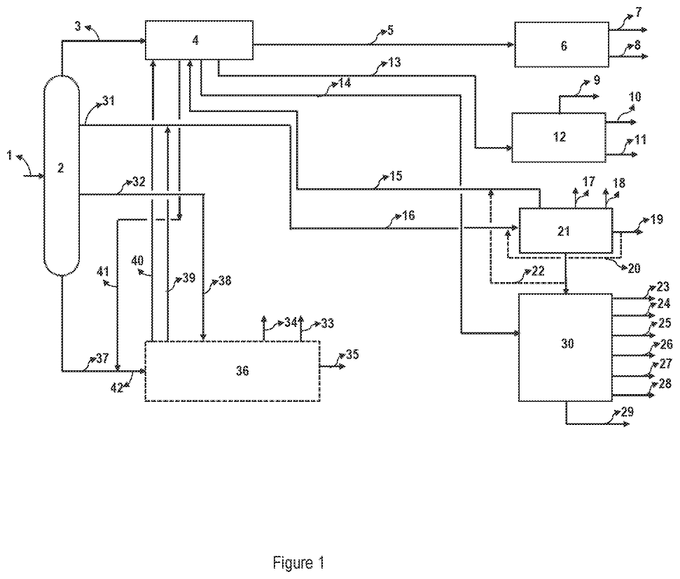

FIG. 1 is the simplified process flow diagram of the integrated crude to chemical complex. In this present process, crude oil is converted to chemical through minimum number of processing steps. Preprocessed crude oil (1) is directly fed into the AVU section (2). The AVU section is consists of atmospheric and vacuum distillation unit. Crude oil is directly fed into atmospheric distillation column and lighter part is fractionated from atmospheric column. The heavier part is called further distilled in vacuum distillation unit operated under vacuum and further fractionated. Together this operation is conducted in this section and termed as atmospheric vacuum unit (AVU). SRN (3) is the light cut obtained from the fractionation of crude oil and fed into NCU (4). SR-MD (31) is the middle distillate boiling range stream obtained from distillation of crude oil in AVU. This stream is mixed with another middle distillate stream (39) from Residue Hydrocracking section (36) and forms a mixed stream (16) to feed into S-MHC unit. VGO (32) is another stream obtained from AVU section and processed in the Residue Hydrocracking section (36) along with VR (37) and PFO (41) obtained from the AVU and NCU respectively. Residue Hydrocracking section consists of slurry hydrocracking reactor and gas-oil section. In the slurry hydrocracking reactor VR is processed along with PFO (41) produced from steam cracking of naphtha in NCU (4). Residue Hydrocracking section produces total naphtha (40), MD (39), along with LPG (34) and off gas (33) streams. Some part of the unconverted oil (UCO) is used for recycling whereas some part is taken out of the unit as bleed and named as pitch (35). Total Naphtha (40) routed towards NCU (4) whereas MD (39) is routed towards SMHC unit (21). NCU is operated based on various naphtha feed stock obtains from AVU (3), Residue Hydrocracking Section (36) and SMHC unit (21). NCU produces ethylene rich fuel gas stream (5) and routed touted towards C2-splitter (6) to separate ethylene (8) from off gas (7). LPG produced in NCU consists of C3 C4 olefins. This olefin rich mixed stream (13) is routed towards PRU (12) to recover propylene (10), 1-butene (11) from LPG (9). Pyrolysis gasoline (14) is also produced in NCU and the stream highly concentrated with mono-aromatics like BTX. Therefore, this stream is processed further in the aromatic complex (29). SMHC unit is a middle distillate hydrocracking unit operated on middle distillate feed stock from AVU and Residue Hydrocracking unit. In this unit mainly five streams are generated. These streams are off gas (17), LPG (18), LN (15), heavy naphtha (22) and ULSD (19). LN produced in this section is sent to NCU for the production of olefins whereas heavy naphtha (22) is sent towards aromatic complex (30). In one of the features of the present invention, a certain part of the unconverted diesel (20) is recycled back in the reactor to increase process yield. Also, another feature of the process reveals that some part of the heavy naphtha (22) can be routed towards NCU through blending with LN (15) to provide flexibility in choosing product pattern (olefin/aromatics and fuel). Aromatic complex consists of reformer, product quality improvement and other product purification unit. In this complex pyrolysis gasoline and heavy naphtha is further processed and produces streams like hydrogen (23), fuel gas (24), LPG (25), benzene (26), p-xylene (27), raffinate blend stock for gasoline (28) and heavy aromatic blend stock for diesel (29).

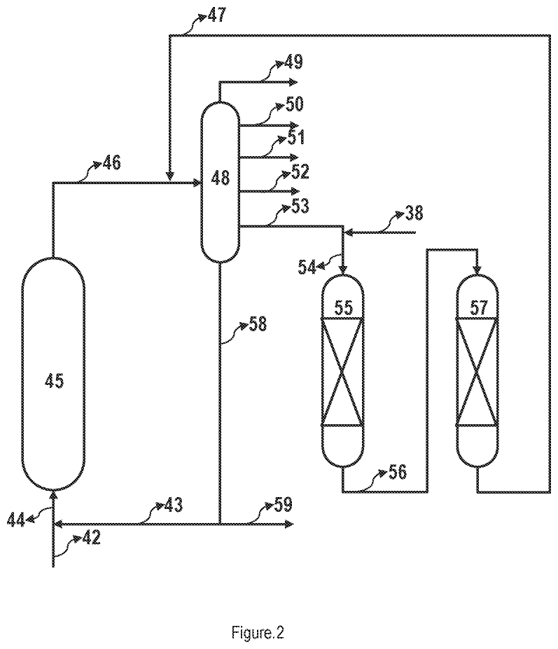

FIG. 2 is the simplified process scheme of Residue Hydrocracking unit. In this section, VR (42) from AVU is mixed with heavy bottom stream (43) from distillation column (48) to produce mixed stream (44). This mixed stream (44) is fed into the slurry reactor (45). The reactor effluent (46) is mixed with gas-oil hydrocracking reactor effluent (47) and fractionated in distillation column (48). Off gas (49), LPG (50), TN (51), MD (52), VGO (53) and unconverted oil (58) is generated through distillation. VGO (54) is mixed with SR-VGO (38) from AVU to produce gas mixed gas oil stream (54) and fed into the hydrotreating reactor (55) of gas-oil hydrocracking unit. Hydrotreated effluent (56) from hydrotreating reactor is fed into hydrocracking reactor (57) and hydrocracked effluent (47) is send back to the distillation column (48). Some part of unconverted oil (43) is recycled back to slurry reactor. Bleed unconverted oil (59) is also called pitch and is taken out of the section as one of the products.

### EXAMPLES

Having described the basic aspects of the present invention, the following non-limiting examples illustrate specific embodiments thereof. Those skilled in the art will appreciate that many modifications may be made in the invention without changing the essence of invention.

### Example 1

Crude blend from an Indian refinery and the properties were analyzed and provided in Table-1.

TABLE 1
Properties of crude blends

| | Crude Blend- |
|---|---|
| Properties | X |
| ° API | 30 |
| CCR, wt % | 5.49 |
| Sulfur, wt % | 1.89 |
| Nitrogen, wt % | 2.5 |
| Asphaltenes, wt % | <0.01 |
| TBP Cut Point | wt % |
| C5- | 3.5 |
| C5-162 | 19 |
| 162-212 | 9.2 |
| 212-349 | 15.3 |
| 249-268 | 3.4 |
| 368-546 | 12.4 |
| 546-562 | 12.4 |
| 562+ | 24.8 |

Crude oil is processed according to the scheme discussed above in FIG. 1 and FIG. 2. However. SMHC unit has been operated on once-through mode. The final product yields out of the complex are provided in Table-2.

TABLE 2
Product yield pattern where SMHC operating in oncethru mode

| | |
|---|---|
| Process Yield | wt % |

Chemical

| | |
|---|---|
| Hydrogen | 2.2 |
| Ethylene | 12.2 |
| Acetylene | 0.1 |
| Propylene | 5.9 |
| Propadiene | 0.3 |
| Butadienes | 1.1 |
| Butenes | 3.5 |
| Benzene | 1.3 |
| Styrene | 0.1 |
| PX | 20.3 |
| Total | 47.1 |
| Chemical | |

Fuel

| | |
|---|---|
| FG | 7.1 |
| LPG | 10.1 |
| Gasoline | 4.6 |
| MD | 18.9 |
| VGO-FO | 1.2 |
| Pitch | 1.0 |
| Fuel & Losses | 7.0 |
| Total Fuel | 42.1 |

### Example 2

In this case, same crude oil is used for the process as in Example-1. The process scheme is also same as Example-1. SMHC unit of this process has been operated on recycle mode. The unconverted part in the SMHC process has been recycled back. Through recycling of the diesel in SMHC unit, p-xylene yield has been increased by 30% along with other chemical product. The chemical yield of the configuration has been improved through this process as presented in Table-3

TABLE 3
Product yield pattern where SMHC operating in Recycle mode

| | |
|---|---|
| Proess Yield | wt % |

Chemical

| | |
|---|---|
| Hydrogen | 2.7 |
| Ethylene | 13.4 |
| Acetylene | 0.2 |
| Propylene | 6.5 |
| Propadiene | 0.3 |
| Butadienes | 1.2 |
| Butenes | 3.9 |
| Benzene | 1.7 |
| Styrene | 0.1 |
| PX | 26.3 |
| Total | 57.9 |
| Chemical | |

Fuel

| | |
|---|---|
| FG | 8.5 |
| LPG | 11.3 |
| Gasoline | 5.3 |
| MD | 6.5 |
| VGO-FO | 1.2 |
| Pitch | 1.0 |
| Fuel & Losses | 7.0 |
| Total Fuel | 42.1 |

### Example-3

In this case. 50% of the heavy naphtha generated in the SMHC process has routed towards NCU to enhance olefin production. SMHC unit has been operated in once through mode and also crude oil used for the process is also same as that of Example-1 & 2.

TABLE 4
Product yield pattern where SMHC operating in Recycle mode

| | |
|---|---|
| Process Yield | wt % |

Chemical

| | |
|---|---|
| Hydrogen | 1.9 |
| Ethylene | 14.4 |
| Acetylene | 0.2 |
| Propylene | 7.0 |
| Propadiene | 0.3 |
| Butadienes | 1.3 |
| Butenes | 4.2 |
| Benzene | 1.0 |
| Styrene | 0.1 |
| PX | 14.7 |
| Total | 45.1 |
| Chemical | |

Fuel

| | |
|---|---|
| FG | 8.5 |
| LPG | 10.8 |
| Gasoline | 4.8 |
| MD | 18.7 |
| VGO-FO | 1.2 |
| Pitch | 1.0 |
| Fuel & Losses | 7.0 |
| Total Fuel | 44.9 |

### Example-4

In this case. LPG produced in the process is further cracked in the Cracker Unit. This may be done in the existing thermal cracker unit/NCU. This will enhance the chemical yield of the process. The yield of the process where SMHC is operating in oncethru mode is in Table-5

TABLE 5
Product yield pattern (SMHC operating in Once thru mode)

| Process Yield | wt % |
|---|---|
| Hydrogen | 2.6 |
| Ethylene | 16.0 |
| Acetylene | 0.2 |
| Propylene | 7.1 |
| Propadiene | 0.3 |
| Butadienes | 1.4 |
| Butenes | 4.2 |
| Benzene | 2.3 |
| Styrene | 0.1 |
| Toluene | 0.6 |
| PX | 22.5 |
| Total | 57.3 |
| Chemical | |
| FG | 10.41 |
| Gasoline | 2.00 |
| MD | 20.97 |
| VGO-FO | 1.23 |
| Pitch | 0.91 |
| Fuel & Losses | 7.22 |
| Total Fuel | 42.74 |

### Example-5

SMHC unit in this example is operating in recycle mode. LPG is processed further in the thermal cracking unit as discussed in Example-4. Rest configuration is kept same as that of Example-4. The process yield is given below in Table-6. This configuration will give the maximum aromatic/PX yield out of the complex.

TABLE 6
Product yield pattern (SMHC operating in Recycle mode)

| Process Yield | wt % |
|---|---|
| Hydrogen | 3.0 |
| Ethylene | 16.9 |
| Acetylene | 0.2 |
| Propylene | 7.5 |
| Propadiene | 0.3 |
| Butadienes | 1.5 |
| Butenes | 4.4 |
| Benzene | 2.7 |
| Styrene | 0.1 |
| Toluene | 0.6 |
| PX | 28.3 |
| Total | 65.6 |
| Chemical | |
| FG | 11.5 |
| Gasoline | 6.3 |
| MD | 7.2 |
| VGO-FO | 1.2 |
| Pitch | 0.9 |
| Fuel & Losses | 7.2 |
| Total Fuel | 34.4 |

### Example-6

SMHC unit in this example is operating in recycle mode. 40% of the HN produced in the SMHC unit is further routed to NCU unit. Also, LPG is processed further in the thermal cracking unit as discussed in Example-4. This is the optimized yield to maximize the chemical yield and also to maximize olefin yield of the process.

TABLE 6
Product yield pattern (40% SMHC-HN to NCU)

| Process Yield | wt % |
|---|---|
| Hydrogen | 2.8 |
| Ethylene | 21.8 |
| Acetylene | 0.2 |
| Propylene | 9.7 |
| Propadiene | 0.4 |
| Butadienes | 2.0 |
| Butenes | 5.7 |
| Benzene | 2.4 |
| Styrene | 0.1 |
| Toluene | 0.8 |
| PX | 20.7 |
| Total | 66.8 |
| Chemical | |
| FG | 15.0 |
| Gasoline | 2.0 |
| MD | 6.9 |
| VGO-FO | 1.2 |
| Pitch | 0.9 |
| Fuel & Losses | 7.2 |
| Total Fuel | 33.2 |

We claim:

1. An integrated process for converting crude oil to high value petrochemicals products, comprising:
i. fractionating crude oil in an atmospheric vacuum unit (AVU) comprising an atmospheric distillation column and a vacuum distillation column to obtain straight run fractions comprising, a straight run naphtha (SRN) having boiling range of C5-200° ° C., a middle distillate stream having boiling range of 200 to 370° C., a vacuum gas oil (VGO) with boiling range of 370 to 560° C. and a vacuum residue (VR) having boiling range above 560° C.;
ii. routing vacuum residue to a Residue Hydrocracking section comprising a slurry hydrocracking reactor and a gas-oil reactor to obtain a stream comprising fuel gas in the range of C1 and C2), liquefied petroleum gas (LPG) in the range of C3 and C4, total naphtha having boiling point in the range of C5 to 200° ° C., middle distillate having boiling point in the range of 200 to 370° C. and unconverted Pitch having boiling point above 560° C. by slurry hydrocracking;
iii. routing the total naphtha towards a naphtha cracker unit for production of olefins;
iv. routing the middle distillate stream towards a selective mild hydrocracking unit;
v. routing the VGO partly towards the gas-oil reactor of the Residue Hydrocracking section;
vi. routing the middle distillate stream from atmospheric distillation unit towards a selective mild hydrocracking unit to obtain a stream comprising fuel gas, LPG, light naphtha having boiling range between C5-100° C., heavy naphtha having boiling range between 100 to 200° C. and diesel stream having boiling point above 200° ° C.;
vii. routing the light naphtha towards a naphtha cracker unit;
viii. routing the heavy naphtha towards an aromatic complex comprising reformer, transalkylation unit, isomer unit, p-xylene extraction unit and aromatic extraction unit;
ix. processing the heavy naphtha in the aromatic complex to obtain hydrogen gas, off gas, LPG, non-aromatic raffinate, benzene, p-xylene and heavy aromatics;
x. routing the SRN towards a naphtha cracker unit to obtain a stream comprising ethylene rich fuel gas stream, C3 and C4 olefin rich mixed stream, monoaromatic rich pyrolysis gasoline stream and pyrolysis fuel oil stream;
xi. routing the ethylene rich fuel gas stream towards a C2-splitter unit to obtain ethylene and fuel gas;
xii. routing the C3 and C4 olefin rich mixed stream towards a propylene recovery unit to obtain propylene, 1-butene, butadiene and LPG;
xiii. routing the monoaromatic rich pyrolysis gasoline stream towards the aromatic complex to obtain benzene and p-xylene;
xiv. routing the pyrolysis fuel oil stream towards Residue Hydrocracking section to crack further.

1. The integrated process as claimed in claim 1, wherein crude oil is pre-processed in one or more unit like, desalter, oil-water separator, gas oil separator to remove impurities for further processing.

2. The integrated process as claimed in claim 1, wherein the atmospheric distillation unit operates at a pressure in the range of 1 to 2 bar (g) and top temperature in the range of 150 to 250° C. and the vacuum distillation unit operates at pressure in the range of 0.01 to 0.05 bar(g).

3. The integrated process as claimed in claim 1, wherein the naphtha cracker unit operates at a reactor outlet temperature of 580 to 670° C., reactor pressure in the range of 0.7 to 2.5 bar(g), and the catalyst to oil ratio in the range of 15 to 30.

4. The integrated process as claimed in claim 1, wherein the Residue Hydrocracking unit consists of a slurry hydrocracker unit which operates at a pressure in the range of 45 to 180 bar(g) and temperature in the range of 360 to 450° C.

5. The integrated process as claimed in claim 1, wherein the residue hydrocracking unit comprising VGO hydrocracker is two reactor system which operates at a pressure in the range of 45 to 140 bar(g) and temperature in the range of 360 to 450° C.

6. The integrated process as claimed in claim 1, wherein the selective mild hydrocracking unit is a two-reactor system operating at a pressure in the range of 45 to 100 bar(g) and temperature in the range of 350 to 420° C.

7. The integrated process as claimed in claim 1, wherein the part of diesel stream produced from the selective mild hydrocracking unit is recycled back into the first reactor of the unit with a fresh feed to recycle ratio of 90:10.

8. The integrated process as claimed in claim 1, wherein the part of heavy naphtha stream produced from the selective mild hydrocracking unit is sent towards the naphtha cracker unit.

9. The integrated process as claimed in claim 1, wherein the whole SR-VGO stream produced from the AVU is hydrocracked in the hydrocracker present in the residue hydrocracking section to produce naphtha and middle distillate.

10. The integrated process as claimed in claim 1, wherein the VGO hydrocracker present in the residue hydrocracking section co-process VGO stream produced from the AVU along with ex-slurry VGO.

11. The integrated process as claimed in claim 1, wherein the naphtha stream produced from the VGO hydrocracker unit has sulfur content <0.5 ppmw.

12. The integrated process as claimed in claim 1, wherein the naphtha stream produced from the selective mild hydrocracking unit has sulfur content <0.5 ppmw.

13. The integrated process as claimed in claim 1, wherein middle distillate stream is processed to produce light naphtha and heavy naphtha stream for production of fuel or petrochemicals.

14. The integrated process as claimed in claim 1, wherein at least 45% of crude oil is converted into chemicals.

15. The integrated process as claimed in claim 1, wherein heavy naphtha produced in the selective mild hydrocracking unit have N+2A value of at least 70 units.

16. An integrated system for converting crude oil to high value petrochemicals products, comprising:
i. an atmospheric vacuum unit (AVU) comprising an atmospheric distillation column and a vacuum distillation column to fractionate crude oil into straight run fractions comprising, a straight run naphtha (SRN) having boiling range of C5-200° C., a middle distillate stream having boiling range of 200 to 370° C., a vacuum gas oil (VGO) with boiling range of 370 to 560° C. and a vacuum residue (VR) having boiling range above 560° C.;
ii. a Residue Hydrocracking section comprising a slurry hydrocracking reactor and a gas-oil reactor to receive and process the vacuum residue to produce a stream comprising fuel gas in the range of C1 and C2), liquefied petroleum gas (LPG) in the range of C3 and C4, total naphtha having boiling point in the range of C5 to 200° C., middle distillate having boiling point in the range of 200 to 370° ° C. and unconverted Pitch having boiling point above 560° C. by slurry hydrocracking, wherein,
a naphtha cracker unit operable to routing the total naphtha for production of olefins;
a selective mild hydrocracking unit operable to routing the middle distillate stream; and
the gas-oil reactor of the Residue Hydrocracking section operable to partly routing the VGO,
iii. a selective mild hydrocracking unit to receive the middle distillate stream from the atmospheric distillation unit and process the said stream to produce a stream comprising fuel gas, LPG, light naphtha having boiling range between C5-100° C., heavy naphtha having boiling range between 100 to 200° ° C. and diesel stream having boiling point above 200° ° C., wherein, the light naphtha is routed towards a naphtha cracker unit;
iv. an aromatic complex comprising reformer, transalkylation unit, isomer unit, p-xylene extraction unit and aromatic extraction unit, wherein the aromatic complex receives heavy naphtha and process said heavy naphtha to produce hydrogen gas, off gas, LPG, non-aromatic raffinate, benzene, p-xylene and heavy aromatics;
v. a naphtha cracker unit to receive SRN and process said SRN to produce a stream comprising ethylene rich fuel gas stream, C3 and C4 olefin rich mixed stream, monoaromatic rich pyrolysis gasoline stream and pyrolysis fuel oil stream, wherein,
the aromatic complex operable to routing the monoaromatic rich pyrolysis gasoline stream to produce benzene and p-xylene;
Residue Hydrocracking section operable to routing the pyrolysis fuel oil stream to crack further.
vi. a C2-splitter unit to receive the ethylene rich fuel gas stream and process the said stream to produce ethylene and fuel gas; and
vii. a propylene recovery unit to receive the C3 and C4 olefin rich mixed stream and process the said stream to produce propylene, 1-butene, butadiene and LPG.
